(19) United States
(12) Patent Application Publication
(10) Pub. No.: US 2005/0260096 A1
(43) Pub. Date: Nov. 24, 2005

(54) Method and apparatus for vaporizing a sterilant fluid using microwave energy

(75) Inventors: Stanley M. Voyten, Albion, PA (US); Michael A. Bacik, Fairview, PA (US); Peter R. Adams, Meadville, PA (US); Michael A. Centanni, Parma, OH (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 10/848,186

(22) Filed: May 18, 2004

(51) Int. Cl. A61L 2/00

(52) U.S. Cl. 422/21

(57) ABSTRACT

A method and apparatus for vaporizing a sterilant fluid using microwave energy. A sterilant fluid is atomized to produce a spray, mist or fog of sterilant fluid. The atomized sterilant fluid is then exposed to microwave energy produced by a microwave generator. Molecules of at least one chemical component of the sterilant fluid rotate in response to exposure to the microwave energy, thereby vaporizing the sterilant fluid.

### FIELD OF THE INVENTION

The present invention relates generally to vaporization of a steriliant fluid, and more particularly to a method and apparatus for vaporizing a sterilant fluid using microwave energy.

### BACKGROUND OF THE INVENTION

Articles are commonly sterilized or decontaminated by exposure to vaporized sterilants. In the prior art, it is well known to vaporize a liquid sterilant by metering liquid sterilant onto a hot surface. The hot surface heats the liquid sterilant, thereby producing a vaporized sterilant. This approach to vaporization has many drawbacks. For instance, considerable time may be needed in order to heat the hot surface to the desired temperature. Furthermore, this type of vaporization system requires an energy consuming high wattage heater.

The present invention overcomes these and other drawbacks of the prior art, and provides a vaporization system that uses a source of microwave energy to vaporize a sterilant fluid in a vaporization chamber, and thereby produce a vapor suitable for use in a sterilization or decontamination process.

### SUMMARY OF THE INVENTION

In accordance with the present invention, there is provided a method for vaporizing a sterilant fluid in a sterilization system, comprising the steps of: (a) atomizing the sterilant fluid, wherein said sterilant fluid is comprised of molecules of at least a first chemical component; and (b) exposing the atomized sterilant fluid to microwave energy having a first frequency to vaporize the sterilant fluid, wherein the molecules of the first chemical component rotate in response to the microwave energy having the first frequency.

In accordance with another aspect of the present invention, there is provided a vaporization system for vaporizing a sterilant fluid in a sterilization system, the vaporization system comprising: (a) means for atomizing the sterilant fluid, wherein said sterilant fluid is comprised of molecules of at least a first chemical component; and (b) a first microwave generator for producing microwave energy having a first frequency to vaporize the sterilant fluid, wherein the molecules of the first chemical component rotate in response to the microwave energy having the first frequency.

In accordance with still another aspect of the present invention, there is provided a method for vaporizing a sterilant fluid comprised of a sterilant component and a carrier component, comprising the steps of: (a) atomizing the sterilant fluid, wherein at least one of said sterilant component and said carrier component is comprised of molecules having a net electrical dipole moment responsive to radiation; and (b) exposing the atomized sterilant fluid to radiation having a first frequency to vaporize the sterilant fluid, wherein said molecules rotate in response to the radiation having the first frequency.

An advantage of the present invention is the provision of a vaporization method and apparatus that more efficiently vaporizes a sterilant fluid than a conventional thermal heating system.

Another advantage of the present invention is the provision of a vaporization method and apparatus that can be easily scaled to vaporize sterilant fluids of varying volumes.

A still further advantage of the present invention is the provision of a vaporization method and apparatus that can selectively excite molecules of a multicomponent sterilant fluid with microwave energy.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a block diagram of a vaporization system according to a preferred embodiment of the present invention.

### DETAILED DESCRIPTION OF A PREFERRED EMBODIMENT

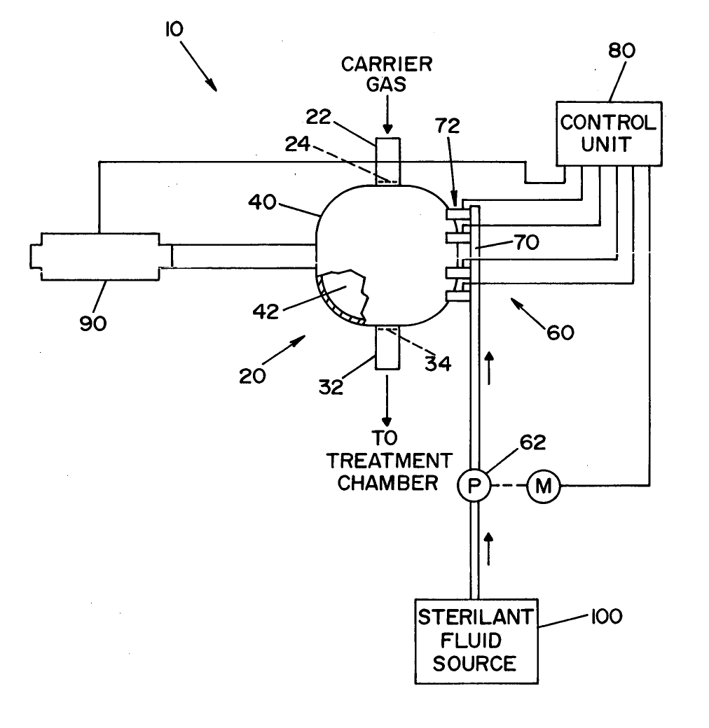

Referring now to the drawing wherein the showing is for the purpose of illustrating a preferred embodiment of the invention only and not for the purpose of limiting same, FIG. 1 shows a block diagram of a vaporization system 10 according to a preferred embodiment of the present invention. It should be understood that the terms sterilant (sterilization) and decontaminant (decontamination) are used interchangeably herein.

Vaporization system 10 is generally comprised of a vaporizer assembly 20, an injection system 60 and a microwave generator 90. Vaporizer assembly 20 includes an inlet conduit 22, an outlet conduit 32 and a housing 40. Housing 40 defines a vaporization chamber 42. Inlet conduit 22 provides a passageway for a carrier gas to enter vaporization chamber 42. Outlet conduit 32 provides a passageway for the carrier gas, and vaporized fluids to exit vaporization chamber 42, as will be described in detail below. Outlet conduit 32 is in fluid communication with a treatment chamber or region (not shown), where articles are exposed to the vaporized fluids to effect sterilization/decontamination of the articles. A blower or fan (not shown) is operable to convey the carrier gas through vaporization chamber 42.

In the illustrated embodiment, vaporizer assembly 20 also includes an inlet screen 24 associated with inlet conduit 22, and an outlet screen 34 associated with outlet conduit 32. Inlet screen 24 and outlet screen 34 act as filters to remove particles from fluids flowing therethrough.

In accordance with the illustrated embodiment, injection system 60 is generally comprised of an injection manifold 70, a plurality of injectors 72, a control unit 80, and a pump 62. Injection system 60 atomizes a sterilant fluid to produce a spray, mist or fog of sterilant fluid, as will be described in detail below.

Manifold 70 is comprised of an inlet conduit that leads to a plurality of outlet conduits. An injector 72 is respectively provided at each of the outlet conduits of manifold 70. Operation of injector 72 is controlled by control unit 80. The inlet conduit of manifold 70 is in fluid communication with pump 62. Pump 62 pumps sterilant fluid from a sterilant fluid source 100 into manifold 70. In a preferred embodiment, pump 62 pressurizes the sterilant fluid to a suitable pressure.

Injector 72 is preferably a conventional liquid injector, such as those used in combustion engines. When injector 72 is energized, an electromagnet moves a plunger that opens a valve in injector 72. This allows pressurized sterilant fluid to squirt out through a small nozzle. The nozzle atomizes the sterilant fluid to produce a fine spray or mist of sterilant fluid. Control unit 80 energizes and de-energizes injectors 72, thereby opening and closing the valves of injectors 72.

Microwave generator 90 provides a source of microwave energy. Microwave generator 90 may be operated in a pulsed mode to provide pulses of microwave energy. Microwaves have wavelengths approximately in the range of 30 cm (corresponding to a frequency of 1 GHz) to 1 mm (corresponding to a frequency of 300 GHz). In accordance with a preferred embodiment, microwave generator 90 takes the form of a magnetron. A magnetron is a high-powered vacuum tube that generates coherent microwaves. The vacuum tube includes a hot filament charged by direct current, built into a resonant cavity and the whole assembly placed in a magnetic field, which deflects the electrons boiling off of the filament, adding energy to the cavity.

It should be appreciated that microwave generator 90 may take alternative forms, including, but not limited to, a klystron or a maser. A maser is a device similar to a laser, except that it works at microwave frequencies.

Operation of vaporization system 10 will now be described in detail. Pump 62 is activated to pressurize sterilant fluid from sterilant fluid source 100. The sterilant fluid includes at least one sterilant or decontaminant chemical component, as will be described in detail below. Control unit 80 energizes injectors 72 to release sterilant fluid therefrom, thereby releasing an atomized spray, mist or fog into vaporization chamber 42. Inside vaporization chamber 42, the atomized spray, mist or fog of sterilant fluid is exposed to microwaves produced by microwave generator 90. Microwave generator 90 is “tuned” to produce microwave energy that will vaporize the sterilant fluid, as will be described in detail below.

A carrier gas (e.g., air), flows into vaporization chamber 42 through inlet conduit 22. The vaporized sterilant fluid produced inside vaporization chamber 42 is conveyed out of vaporization chamber 42 through outlet conduit 32. Outlet conduit 32 is in fluid communication with the treatment chamber (not shown), where articles are exposed to the vaporized sterilant fluid to effect sterilization or decontamination thereof.

The sterilant fluid may be comprised of two or more chemical components, namely, a sterilant component and a carrier component. The sterilant component is an active chemical for a sterilization or decontamination process. The carrier component is a fluid that may act as a diluent for the sterilant component. It should be understood that the carrier component may also be an active chemical for the sterilization or decontamination process.

Common sterilant components include, but are not limited to, liquid hydrogen peroxide, peracids such as peracetic acid, and bleach. It is also contemplated that the sterilant component may be a gas, including, but not limited to, ozone, chlorine dioxide, and ethylene oxide. Common carrier components include, but are not limited to water, de-ionized water, distilled water, an alcohol (e.g., a tertiary alcohol), peroxide, a glycol-containing chemical compound, and combinations thereof. Glycol-containing chemical compounds include, but are not limited to, polyethylene glycol, diethylene glycol, triethylene glycol, tetraethylene glycol, glycol ethers, polypropylene glycol, propylene glycol, and combinations thereof. It should be appreciated that the above-identified liquid sterilant components (e.g., hydrogen peroxide) may also serve as a carrier component.

Some typical combinations of sterilant components and carrier components include, but are not limited to, hydrogen peroxide and water, bleach and water, peracetic acid and water, hydrogen peroxide and an alcohol, an alcohol and water, and ozone and water.

Molecules having net electrical dipole moments may be excited by microwave radiation of a specific frequency or frequencies. In this regard, the microwave radiation impinging upon a molecule having a net electric dipole moment will exert a torque on the molecule. The oscillating electric field of the applied radiation tries to align the electric dipole moment along the electric field's axis. The electric field of the microwave radiation continually changes in its magnitude and direction, thus rotating the dipole moment and hence the molecule. It should be understood that some molecules having electrical dipole moments may be excited by infrared radiation of a specific frequency or frequencies.

It should be appreciated that the ease in which an electric field can rotate a molecule will vary depending upon properties of the molecule, such as the moment of inertia (I). Moment of inertia (I) can be expressed as follows:
I=Σmᵢ(rᵢ)²,
where mᵢ is the mass of the molecule, and rᵢ is the distance to atoms of the molecule.

Taking, for example, a sterilant fluid comprised of hydrogen peroxide and water, a hydrogen peroxide molecule has nearly twice the mass of a water molecule, and has two oxygen atoms separated by a distance of 0.149 nm. Specifically, the hydrogen peroxide molecule has a moment of inertia (I) of about 34×10⁻⁴⁰ (grams)(cm²), while the water molecule has a moment of inertia (I) of about 1.1×10⁻⁴⁰ (grams)(cm²). Thus, the moment of inertia (I) of the hydrogen peroxide molecule is about 34 times greater than the moment of inertia (I) of the water molecule. Due to the smaller moment of inertia (I) for the water molecule, it is easier to rotate the water molecule with microwave energy than it is to rotate the hydrogen peroxide molecule with microwave energy. Moreover, in a droplet form, it is easier for the water molecules to rotate than it is for hydrogen peroxide molecules to rotate, because of the dumb-bell like structure of the hydrogen peroxide molecule.

It should be understood that the foregoing description excludes the effects of hydrogen bonding that plays a part in the mechanism of energy transfer to each molecule. Namely, the foregoing description considers bombarding a single water molecule and a single hydrogen peroxide molecule in a region of space with microwave radiation.

As indicated above, microwave generator 90 is “tuned” to produce microwaves that will vaporize the sterilant fluid. In one embodiment of the present invention, microwave generator 90 may be tuned to produce microwaves having a frequency that “excites” molecules of the sterilant component. In a second embodiment of the present invention, microwave generator 90 may be tuned to produce microwaves having a frequency that “excites” molecules of the carrier component. In yet another embodiment of the present invention, microwave generator 90 may produce microwaves that alternate between a first frequency that excites molecules of the sterilant component and a second frequency that excites molecules of the carrier component. In still another embodiment, two microwave generators are used simultaneously. In this regard, microwave generator 90 produces microwaves of a first frequency that excites molecules of the sterilant component, while a second microwave generator simultaneously produces microwaves of a second frequency that excites molecules of the carrier component.

As the atomized sterilant fluid is bombarded with microwave radiation, the molecules that are “excited” by the frequency of the microwaves will essentially be “driven” or “boiled” away from any unexcited molecules, as the dipole moments of excited molecules are rotated. Accordingly, both the excited and unexcited molecules are released as a vapor. Kinetic energy will also be imparted to the unexcited molecules as the excited molecules bump into the unexcited molecules, thus facilitating vaporization of the excited and unexcited molecules.

It should be understood that the present invention may be used to vaporize a sterilant fluid where the sterilant component and/or the carrier component of the sterilant fluid is comprised of molecules that have a net electrical dipole moment that allows absorption of microwave radiation of a specific frequency or frequencies. Accordingly, only the sterilant component or the carrier component of the steriliant fluid needs to have a net electrical dipole moment that allows absorption of microwave radiation of a specific frequency or frequencies. In this regard, a suitable sterilant fluid for use in connection with the present invention may be comprised of a sterilant component having molecules that are not excitable by microwave radiation, and a carrier component having molecules that are excitable by microwave radiation, or vice versa.

Water molecules will absorb microwave energy at a frequency of about 2.450 GHz. Approximate microwave absorption frequencies for hydrogen peroxide molecules are provided in the table below:

Microwave Absorption Spectrum
for Hydrogen Peroxide

14.829 GHz
37.518 GHz
22.054 GHz
27.640 GHz
11.072 GHz
35.916 GHz
39.033 GHz
39.495 GHz
39.790 GHz

It should be appreciated that while the present invention has been described with reference to a sterilant fluid comprised of a sterilant component and a carrier component, it also contemplated that the present invention may be used in connection with a sterilant fluid comprised solely of a sterilant component. The sterilant component is atomized as described above, and then exposed to microwaves having a frequency that excites the molecules of the sterilant component, thereby vaporizing the sterilant component.

Other modifications and alterations will occur to others upon their reading and understanding of the specification. It is intended that all such modifications and alterations be included insofar as they come within the scope of the invention as claimed or the equivalents thereof.

1. A method for vaporizing a sterilant fluid in a sterilization system, comprising the steps of:
atomizing the sterilant fluid, wherein said sterilant fluid is comprised of molecules of at least a first chemical component; and
exposing the atomized sterilant fluid to microwave energy having a first frequency to vaporize the sterilant fluid, wherein the molecules of the first chemical component rotate in response to the microwave energy having the first frequency.

2. A method according to claim 1, wherein said first ch-irLical component is a sterilant component.

3. A method according to claim 2, wherein said sterilant component is selected from the group consisting of: hydrogen peroxide, a peracid, and bleach.

4. A method according to claim 1, wherein said first chemical component is a carrier component.

5. A method according to claim 4, wherein said carrier component is selected from the group consisting of: water, de-ionized water, distilled water, an alcohol, peroxide, a glycol-containing chemical compound, and combinations thereof.

6. A method according to claim 5, wherein said glycol-containing chemical compound is selected from the group consisting of: polyethylene glycol, diethylene glycol, triethylene glycol, tetraethylene glycol, glycol ethers, polypropylene glycol, propylene glycol, and combinations thereof.

7. A method according to claim 1, wherein said sterilant fluid is comprised of molecules of the first chemical component, and molecules of a second chemical component.

8. A method according to claim 7, wherein said first chemical component is a sterilant component, and the second chemical component is a carrier component.

9. A method according to claim 7, wherein said first chemical component is a carrier component, and the second chemical component is a sterilant component.

10. A method according to claim 7, wherein said method further comprises:
exposing the atomized sterilant fluid to microwave energy having a second frequency, wherein the molecules of the second chemical component rotate in response to the microwave energy having the second frequency.

11. A method according to claim 10, wherein said microwave energy having the first frequency is simultaneously produced with said microwave energy having the second frequency.

12. A method according to claim 10, wherein said microwave energy having the first frequency is alternately produced with said microwave energy having the second frequency.

13. A method according to claim 1, wherein said first frequency is in a range of 1 GHz to 300 GHz.

14. A method according to claim 13, wherein said first frequency is about 2.450 GHz.

15. A method according to claim 13, wherein said first frequency is selected from the group of frequencies consisting of: about 14.829 GHz, about 37.518 GHz, about 22.054 GHz, about 27.640 GHz, about 11.072 GHz, about 35.916 GHz, about 39.033 GHz, about 39.495 GHz, and about 39.790 GHz.

16. A method according to claim 1, wherein said microwave energy is pulsed.

17. A vaporization system for vaporizing a sterilant fluid in a sterilization system, the vaporization system comprising:
means for atomizing the sterilant fluid, wherein said sterilant fluid is comprised of molecules of at least a first chemical component; and
a first microwave generator for producing microwave energy having a first frequency to vaporize the sterilant fluid, wherein the molecules of the first chemical component rotate in response to the microwave energy having the first frequency.

18. A vaporization system according to claim 17, wherein said first chemical component is a sterilant component.

19. A vaporization system according to claim 18, wherein said sterilant component is selected from the group consisting of: hydrogen peroxide, a peracid, and bleach.

20. A vaporization system according to claim 17, wherein said first chemical component is a carrier component.

21. A vaporization system according to claim 20, wherein said carrier component is selected from the group consisting of: water, de-ionized water, distilled water, an alcohol, peroxide, a glycol-containing chemical compound, and combinations thereof.

22. A vaporization system according to claim 21, wherein said glycol-containing chemical compound is selected from the group consisting of: polyethylene glycol, diethylene glycol, triethylene glycol, tetraethylene glycol, glycol ethers, polypropylene glycol, propylene glycol, and combinations thereof.

23. A vaporization system according to claim 17, wherein said sterilant fluid is comprised of molecules of the first chemical component, and molecules of a second chemical component.

24. A vaporization system according to claim 23, wherein said first chemical component is a sterilant component, and the second chemical component is a carrier component.

25. A vaporization system according to claim 23, wherein said first chemical component is a carrier component, and the second chemical component is a sterilant component.

26. A vaporization system according to claim 23, wherein said method further comprises:
a second microwave generator for producing microwave energy having a second frequency, wherein the molecules of the second chemical component rotate in response to the microwave energy having the second frequency.

27. A vaporization system according to claim 26, wherein said microwave energy having the first frequency is simultaneously produced with said microwave energy having the second frequency.

28. A vaporization system according to claim 23, wherein said first microwave generator produces microwave energy having a second frequency, wherein the molecules of the second chemical component rotate in response to the microwave energy having the second frequency energy, said microwave energy having the first frequency is alternately produced with said microwave energy having the second frequency.

29. A vaporization system according to claim 17, wherein said first frequency is in a range of 1 GHz to 300 GHz.

30. A vaporization system according to claim 29, wherein said first frequency is about 2.450 GHz.

31. A vaporization system according to claim 29, wherein said first frequency is selected from the group of frequencies consisting of: about 14.829 GHz, about 37.518 GHz, about 22.054 GHz, about 27.640 GHz, about 11.072 GHz, about 35.916 GHz, about 39.033 GHz, about 39.495 GHz, and about 39.790 GHz.

32. A vaporization system according to claim 17, wherein said microwave energy generated by said first microwave generator is pulsed.

33. A method for vaporizing a sterilant fluid comprised of a sterilant component and a carrier component, comprising the steps of:
atomizing the sterilant fluid, wherein at least one of said sterilant component and said carrier component is comprised of molecules having a net electrical dipole moment responsive to radiation; and
exposing the atomized sterilant fluid to radiation having a first frequency to vaporize the sterilant fluid, wherein said molecules rotate in response to the radiation having the first frequency.

34. A method according to claim 33, wherein only said sterilant component is comprised of molecules having a net electrical dipole moment responsive to radiation.

35. A method according to claim 33, wherein only said carrier component is comprised of molecules having a net electrical dipole moment responsive to radiation.

36. A method according to claim 33, wherein said radiation is microwave radiation.

37. A method according to claim 33, wherein said radiation is infrared radiation.
